(19) United States
(12) Patent Application Publication
(10) Pub. No.: US 2018/0060501 A1
(43) Pub. Date: Mar. 1, 2018

(54) SYSTEM AND METHOD FOR GENERATING CLINICAL ACTIONS IN A HEALTHCARE DOMAIN

(71) Applicant: Wipro Limited, Bangalore (IN)

(72) Inventor: Abhishek Gunjan, Gaya (IN)

(21) Appl. No.: 15/299,304

(22) Filed: Oct. 20, 2016

(30) Foreign Application Priority Data
Aug. 30, 2016 (IN) 201641029594

(51) Int. Cl. G06F 19/00; G06F 17/30

(52) U.S. Cl. CPC G06F 19/324; G06F 19/3456; G06F 19/345; G06F 17/30867; G06F 17/30598; G06F 17/3053; G06F 19/322

(57) ABSTRACT

Systems and methods for generating one or more actions are disclosed. The system retrieves data associated with patients from data sources. The data is analyzed to classify the patients into different categories. The system generates a set of profiles for the patients based on the data. A plurality of clusters is also generated based on the classification of the patients and the set of profiles. The system generates trend model based on the plurality of clusters. The trend comprises trend of plurality of diseases and rate of recovery of the plurality of diseases based on existing procedures and medications applied. Based on the trend model, the system generates scores corresponding to the plurality of clusters. Further, the clusters are ranked based on their scores. Finally, the system generates one or more actions (i.e., clinical actions) which include new procedure and a new medication based on the ranking of the clusters.

This application claims the benefit of Indian Patent Application Serial No. 201641029594 filed Aug. 30, 2016, which is hereby incorporated by reference in its entirety.

### FIELD

The present disclosure relates in general to data processing. More particularly, but not exclusively, the present disclosure discloses a method and system for generating clinical actions in a healthcare domain.

### BACKGROUND

In this era of digital and automated world, clinical decision support system (CDSS) is an important breakthrough in a healthcare domain. The healthcare world has witnessed pre-CDSS and post-CDSS period. During the pre-CDSS period when such digitization and automation was not available, healthcare practitioners used to rely on paper-based medical reports for rendering clinical decisions or clinical decision making. The clinical decision making is an ability of the healthcare practitioners to take healthcare related decisions based on medical reports/medical data and their professional experiences. Now, over the time, as the medical data has enormously increased, automated healthcare systems like the CDSS came into existence.

These automated healthcare systems enable the healthcare practitioners to take proper judgement and decisions (clinical decisions) quite effectively than the decision made during the pre-CDSS period. The clinical decisions or judgements are crucial not only because it affects the health of a person/patient, but it also makes the healthcare professionals accountable for their decisions. Thus, to properly render the clinical decisions, it is important that the automated healthcare systems should be up-to-date and advanced with data processing techniques.

Some of major challenges observed in the existing automated healthcare systems are drilling down of the medical data for getting the information at a granular level. The reasons for such challenge are that the existing automated healthcare systems lacks data clustering and data correlations. All these challenges make the existing healthcare systems inefficient because it not only consumes more time and hardware resources, but it also fails to deliver deeper level of information to the healthcare practitioners.

### SUMMARY

Disclosed herein is method and system for generating one or more actions (i.e., clinical actions) in a healthcare domain. Data associated with patients is retrieved from plurality of data sources. The data is analyzed to classify the patients into different categories, for example, predefined disease category and medical finding category. Further, a set of profiles are generated for each patient based on the data retrieved. A plurality of clusters is also generated based on the classification of the patients and the set of profiles. Further, the system generates a trend model based on the plurality of clusters, wherein the trend model reflects a relation between (i.e., trend) a plurality of diseases and a rate of recovery of the plurality of diseases based on existing procedures and existing medications applied. In other words, such type of trend helps the system to understand recovery rate for a particular disease on a group of patients when a particular medication or treatment is applied. Based on the trend model, scores are generated corresponding to the plurality of clusters. Further, the system performs ranking of the plurality of clusters based on the plurality of scores. Finally, the system generates one or more actions (i.e., clinical actions) which include new procedure and a new medication based on the ranking of the clusters. According to an aspect of present disclosure, the clinical actions may be generated based on a search query input by medical practitioner.

Accordingly, the present disclosure relates to a method for generating one or more actions in a healthcare domain. The method comprises the steps of retrieving data comprising patient-specific information and surveyed information from a plurality of data sources. The data is associated with a plurality of patients. Further, the method comprises a step of analyzing the data to classify each of the plurality of patients into a predefined disease category and a medical finding category. The method further comprises generating a set of profiles for each of the plurality of patients based on the data. Further, the method comprises generating a plurality of clusters based on the classification of each of the plurality of patients and the set of profiles. Each cluster comprises two or more patients having similarity in at least one of the predefined disease category, the medical finding category, and the set of profiles. The method further comprises generating a trend model based on the plurality of clusters. The trend model comprises a trend of a plurality of diseases and a rate of recovery of the plurality of diseases based on existing procedures and existing medications associated with each of the plurality of the patients. Further, the method comprises generating a plurality of scores corresponding to the plurality of clusters based on the trend model. The method further comprises the step of ranking the plurality of clusters based on the plurality of scores. Further, the method comprises generating one or more actions based on the ranking. The one or more actions comprise a new procedure and a new medication.

Further, the present disclosure relates to a clinical action generating system for generating one or more actions in a healthcare domain. The clinical action generating system comprises a processor and a memory communicatively coupled to the processor. The memory stores processor-executable instructions, which, on execution, causes the processor to perform one or more operations comprising retrieving data comprising patient-specific information and surveyed information from a plurality of data sources. The data is associated with a plurality of patients. The system further analyzes the data to classify each of the plurality of patients into a predefined disease category and a medical finding category. The system further generates set of profiles for each of the plurality of patients based on the data. Further, the system also generates a plurality of clusters based on the classification of each of the plurality of patients and the set of profiles. Each cluster comprises two or more patients having similarity in at least one of the predefined disease category, the medical finding category, and the set of profiles. The system further generates a trend model based on the plurality of clusters. The trend model comprises a trend of a plurality of diseases and a rate of recovery of the plurality of diseases based on existing procedures and existing medications associated with each of the plurality of patients. Further, the system generates a plurality of scores corresponding to the plurality of clusters based on the trend model. The system further ranks the plurality of clusters based on the plurality of scores. Finally, the system generates one or more actions based on the ranking. The one or more actions comprise a new procedure and a new medication.

Further the present disclosure relates to a non-transitory computer-readable storage medium for generating one or more actions in a healthcare domain is disclosed, which when executed by a computing device, cause the computing device to perform operations including retrieving data comprising patient-specific information and surveyed information from a plurality of data sources. The data is associated with a plurality of patients. The operations further analyze the data to classify each of the plurality of patients into a predefined disease category and a medical finding category. The operations further generate set of profiles for each of the plurality of patients based on the data. Further, the operations also generate a plurality of clusters based on the classification of each of the plurality of patients and the set of profiles. Each cluster comprises two or more patients having similarity in at least one of the predefined disease category, the medical finding category, and the set of profiles. The operations further generate a trend model based on the plurality of clusters. The trend model comprises a trend of a plurality of diseases and a rate of recovery of the plurality of diseases based on existing procedures and existing medications associated with each of the plurality of patients. Further, the operations generate a plurality of scores corresponding to the plurality of clusters based on the trend model. The operations further rank the plurality of clusters based on the plurality of scores. Finally, the operations generate one or more actions based on the ranking. The one or more actions comprise a new procedure and a new medication.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 shows an exemplary environment illustrating a clinical action generating system for generating one or more actions in a healthcare domain in accordance with some embodiments of the present disclosure;

FIG. 2 shows a detailed block diagram illustrating the clinical action generating system in accordance with some embodiments of the present disclosure;

FIG. 3 shows a flowchart illustrating a method of generating one or more actions in a healthcare domain in accordance with some embodiments of the present disclosure; and

FIG. 4 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternative falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure relates to a method and a clinical action generating system for generating one or more actions in a healthcare domain. Although, the method for generating the one or more actions is described in conjunction with a server, the said method can also be implemented in various computing systems/devices, other than the server. The one or more actions are the clinical actions which are generated by the clinical action generating system. The clinical actions are generated not only on basis of data extracted from various data sources, but also using different data processing techniques like data clustering and data profiling. All these techniques reduce the overload of internal resources, of the clinical action generating system, like processor's speed and memory utilization. In other words, the clinical action generating system becomes efficient and faster while rendering the clinical decisions i.e., generating the clinical actions.

The clinical action generating system (alternatively referred as system) retrieves data from various data sources. The data retrieved is associated with number of patients. The data indicates patient-specific information and surveyed information. Using the data, the system builds a knowledge database, which is explained in detail in subsequent paragraphs of the specification. The knowledge database serves as a central repository where the data retrieved is stored in a structured format for faster retrieval of useful information required for rendering the clinical decisions. For example, profiling and clustering is performed by the system to structure the data. One of an objective of such structuring is to enable the system to function more efficiently while generating the one or more actions.

The clinical action generating system not only retrieves the information efficiently, but it also has an in-built learning capability which makes the clinical action generating system more robust and advanced. Thus, the disclosed clinical action generating system knows how to delve into finer details based on the available data and ongoing learning for rendering accurate clinical decisions.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

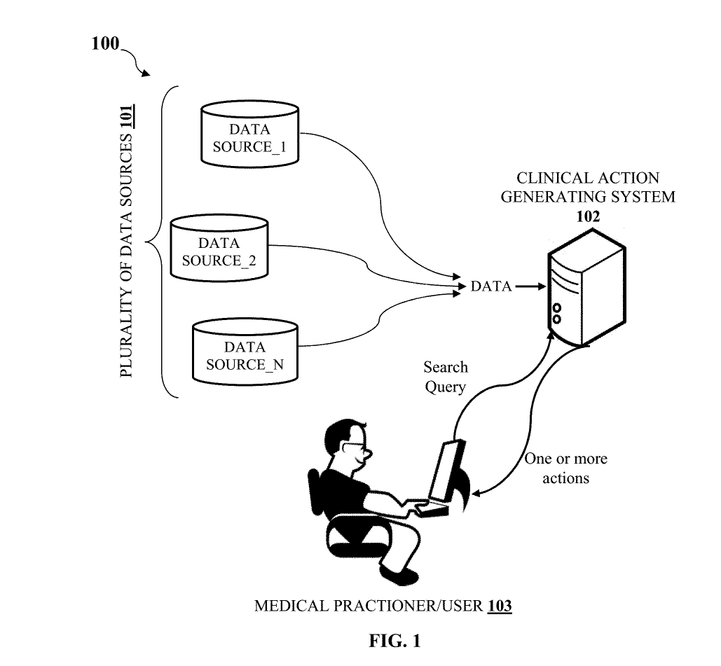

FIG. 1 shows an exemplary environment illustrating a clinical action generating system for generating one or more actions in a healthcare domain.

The environment 100 comprises a plurality of data sources 101, the clinical action generating system 102 and a medical practitioner/user 103 connected with the clinical action generating system 102. The plurality of data sources 101 may be a repository for storing huge amount of data related to the medical or healthcare system. For example, the plurality of data sources 101 comprises at least one of clinical sources, hospice item set (HIS), legacy system, social media, blogs and journals. In an embodiment, the plurality of data sources 101 may be configured with a data sorting technique for sorting and classifying various types of data stored in the plurality of data sources 101. In an embodiment, the plurality of data sources 101 may be placed within the clinical action generating system 102.

The clinical action generating system 102 retrieves data 208 from the plurality of data sources 101 for analysis. Further, the data retrieved is associated with a plurality of patients. The data 208 may comprise, but not limited to, patient-specific information 214 and surveyed information 216. Based on the analysis, the clinical action generating system 102 creates a knowledge database 212. The clinical action generating system 102 further performs data profiling and data clustering for efficiently retrieving useful information. In an embodiment, the clinical action generating system 102 may include, but not limited to, a server, a computer, a workstation, a laptop, mobile phone, or any computing system/device capable of receiving, analysing and processing the useful information.

Further, the clinical action generating system 102 may be accessed by a medical practitioner or a user 103 connected via his/her device. According to embodiments of present disclosure, the medical practitioner may perform a search using a search query to retrieve the useful information i.e., one or more actions generated by the clinical action generating system 102 which is explained in detail in the upcoming paragraphs of the specification.

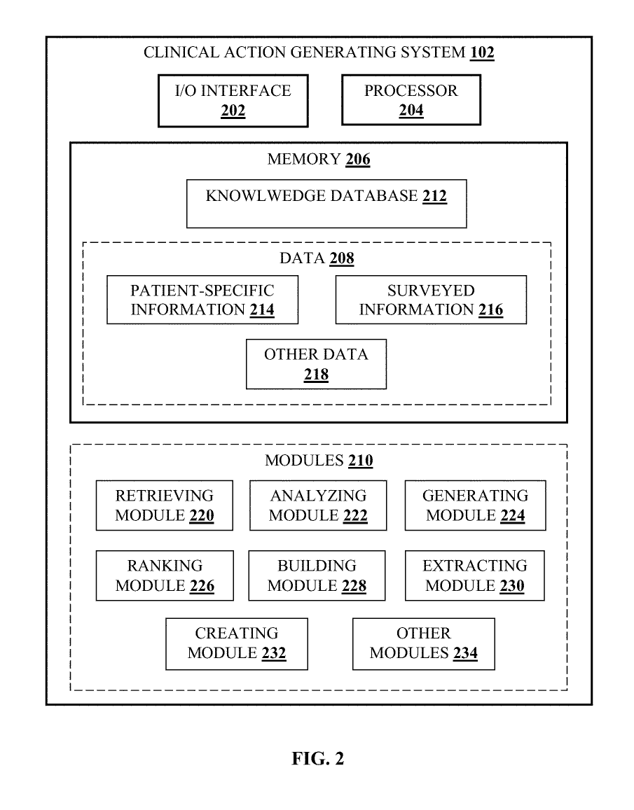

FIG. 2 shows a detailed block diagram illustrating the clinical action generating system in accordance with some embodiments of the present disclosure.

The clinical action generating system 102 comprises an I/O interface 202, a processor 204 and a memory 206. The I/O interface 202 is configured to receive the data 208 from the plurality of data sources 101. The memory 206 is communicatively coupled to the processor 204. The processor 204 is configured to perform one or more functions of the clinical action generating system 102 for generating one or more actions in a healthcare domain. In one implementation, the clinical action generating system 102 comprises data 208 and modules 210 for performing various operations in accordance with the embodiments of the present disclosure. The memory 206 further comprises a knowledge database 212. In an embodiment, the data 208 may include, without limitation, patient-specific information 214, surveyed information 216, and other data 218.

In one embodiment, the data 208 may be stored within the memory 206 in the form of various data structures. Additionally, the aforementioned data 208 can be organized using data models, such as relational or hierarchical data models. The other data 218 may store data, including temporary data and temporary files, generated by the modules 210 for performing the various functions of the clinical action generating system 102.

In an embodiment, the knowledge database 212 is created by analyzing the data 208 retrieved from the plurality of data sources 101. The knowledge database 212 serves as a central repository for storing the data 208 in a structured format.

In an embodiment, the data 208 may be processed by one or more modules 210. In one implementation, the one or more modules 210 may also be stored as a part of the processor 204. In an example, the one or more modules 210 may be communicatively coupled to the processor 204 for performing one or more functions of the clinical action generating system 102.

In one implementation, the one or more modules 210 may include, without limitation, a retrieving module 220, an analyzing module 222, a generating module 224, a ranking module 226, a building module 228, an extracting module 230, a creating module 232, and other modules 234. As used herein, the term module refers to an application specific integrated circuit (ASIC), an electronic circuit, a processor (shared, dedicated, or group) and memory that execute one or more software or firmware programs, a combinational logic circuit, and/or other suitable components that provide the described functionality.

In an embodiment, the retrieving module 220 retrieves the data 208, from the plurality of data sources 101, associated with a plurality of patients. These data sources 101 may comprise, but not limited to, clinical sources, hospice item set (HIS), legacy system, social media, blogs or journals. Although, the aforesaid data sources 101 comprise various type of data, however, the retrieving module 220 retrieves or ingest only clinically/healthcare related data. For example, the retrieving module 220 identifies and retrieves only healthcare related information when interacting with data sources like social media, blogs or journals.

The retrieving module 220 may use inbuilt connectors and Extract, Transform, and Load (ETL) logics during the data 208 retrieval process. The retrieving module 220 may also interact with the data sources 101 like legacy system to ingest data which is stored in different form of data stores or file systems. The data 208 retrieved may comprise patient-specific information 214 and surveyed information 216. For example, the patient-specific information 214 comprises, but not limited to, diagnosis summary, health parameters, or results of a medical test conducted upon the patient. On the other hand, the surveyed information 216 comprises, without limitation, sample size of a population, criticality of patients, post-medication observations or other findings by the research practitioners and doctors.

Further, the patient-specific information 214 retrieved from the data sources 101 may be exchanged between two or more entities like healthcare or medical institutions. For example, it may happen that that a hospital (healthcare institution) might have only partial information about a patient and initial level of findings might be present with some other hospital. To handle such scenario, the retrieving module 220 implements a data exchange policy. This policy helps in the interaction between two or more remotely located hardware components associated with the different healthcare institutions.

In an embodiment, once the data 208 is retrieved, the analyzing module 222 analyzes the data 208 to create different classifications like predefined disease category and medical finding category. Each of the plurality of patients is then classified based on the abovementioned categories. The analyzing module 222 may also implement an ontology builder while creating the classifications. The ontology builder may have an in-built classifiers to help the system 102 understand one or more sub-categories or sub-classifications in order to get into greater detail of the data 208. For example, the other categories or sub-categories may be type of disease, level of illness and the like. It also has learning mechanism which not only matures the system 102 based on past experiences, but on the other hand, it also learns new categories and sub-categories in which the classification of the patients and the data 208 can be performed.

Now since the data 208 retrieved from the data source like social media and blogs, in an embodiment, the generating module 224 generates a set of profiles for each of the plurality of patients based on the data 208. The set of profiles may comprise a persona, a family profile and a genetic profile. For generating the persona of a patient, the generating module 224 extracts socio-economic information from the data 208. This type of information is not readily available in other data sources from which the data 208 is extracted, for example, hospice item set (HIS) and legacy systems. That's why, the generating module 224 extracts the relevant data from the data sources like social media and blogs.

According to embodiments of present disclosure, the generating module 224 also generates the family profile of the patients. Now, while generating the family profile, the generating module 224 focuses on medical data which is associated with the family members of the patient. For example, if the patient has shared a post on the social media (i.e., one of the data sources) in which he/she has tagged his/her family members, the generating module 224 will detect such post and extract the medical related information for generating the family profile. This type of information i.e., the family profile helps the system 102 understand that if certain behavior and observations are more common in the family members, then there could be a likelihood of its occurrence in the patient also.

According to some embodiments of present disclosure, the generating module 224 also generates the genetic profile of the patients. For generating the genetic profile, the genetic sequencing of the disease (defined by the ontology builder) is used. The genetic profiling information helps the system 102 to predict the accurate disease and causes for the patients. The generating module 224 not only generates the genetic profile, but is also learns how genetic sequencing is modified or changed. This learning mechanism helps the system 102 to understand about possible reasons about such modification/changes in the genetic sequencing. Now since the causes may be predicted for the different diseases, an extracting module 230 extracts the key or most relevant etiological features from the data 208.

According to some embodiments, the generating module 224 generates a plurality of clusters based on the classification of the plurality of patients and the set of profiles. The clustering is done to club or collate patients having similarity in their features. For example, a cluster may comprise two or more patients having similarity in the features like the predefined disease category, the medical finding category, and the set of profiles. This helps the system 102 to analyze the patients on similar aspects which many of the time may be either ignored or not considered due to domain limitation at times. Further, the generating module 224 generates the clusters at different levels of the classification. During the clustering, the generating module 224 also learns about the new clusters and topics over the time.

Considering an example of a liver specific data, a medical ontology may contain diseases like Hepatocellular carcinoma (HCC), Carcinoma, Hepatitis B, Hepatitis C, and the like. The first level of classification first classifies the information as whether it is HCC or Carcinoma. However, the same information can also be found in multiple classes like Hepatitis B and Hepatitis C.

According to embodiments, the generating module 224, based on the plurality of clusters, generates a trend model. The trend model comprises a trend of a plurality of diseases and a rate of recovery of the plurality of diseases based on existing procedures and existing medications associated with the plurality of the patients. In other words, the clustering and the trend help the system 102 to understand recovery rate for a particular disease on a similar set of patients when a particular medication or treatment is applied. Further, the generating module 224 generates a plurality of scores corresponding to the plurality of clusters based on the trend model.

Based on the information generated by the generating module 224, a creating module 232 creates a knowledge database 212. For example, the data 208 retrieved from the plurality of sources 101, the set of profiles, the plurality of clusters and the trend model are considered for creating the knowledge database 212. The knowledge database 212 serves a central repository for the system 102 and it is further updated time to time. According to embodiments, the system 102 also comprises a building module 228 for building a learning model based on the data 208 retrieved from the data sources 101 i.e., the patient-specific information 214 and the surveyed information 216.

Now, according to embodiments, a ranking module 226 ranks the plurality of clusters based on the plurality of scores. Based on the ranking, the generating module 224 generates one or more actions comprising a new procedure and a new medication for the patients. For example, when a medical practitioner/user 103 requires assistance while rendering clinical decisions, he/she may form a search query for retrieving important information from the knowledge database 212 of the system 102. The system 102 also enables the medical practitioner/user 103 to form their search query using Boolean operators like AND, OR, NOT, XOR and the like. This helps in further refining the search. According to an example, the system 102 may receive a search query as “Cirrhosis” from the medical practitioner/user 103. However, according to another example, the system 102 may receive a search query like “Cirrhosis AND phtn OR asities” i.e., using Boolean operators. The system 102 executes the search query to retrieve two or more clusters from the plurality of clusters stored in the knowledge database 212. The two or more clusters which are retrieved are related to each other or similar to each other in terms of different medical data. Then, the system 102 correlates the two or more clusters to generate one or more correlated-clusters. The purpose of such correlation is to generate the clusters (i.e., one or more clusters) based on different medical data like symptoms, observations, procedures, medication outcomes, rate of change of test parameters or health parameters. Thus, this helps in rendering the clinical actions in a better manner.

Further, the ranking module 226 may also be used while generating the one or more actions. For example, the ranking module 226 considers the keywords being searched along with other dimensions like age group of the patients, type of doctor currently looking after the patients, total number of doctors, type of doctors who has previously looked after the patients, and commonality of a particular disease in that age group of the patients.

For example, if keywords like “HCC” and “Asities” is searched together, the system 102 analyzes the total number of cases with HCC and Asities and also looks whether it contains other etiology along with learned patterns of age group, gender, etc. in which the occurrence of HCC with Asities is more common while ranking. As stated above, the dimensions like number of doctors and type of doctor who has looked after the patients is also considered while generating the one or more actions.

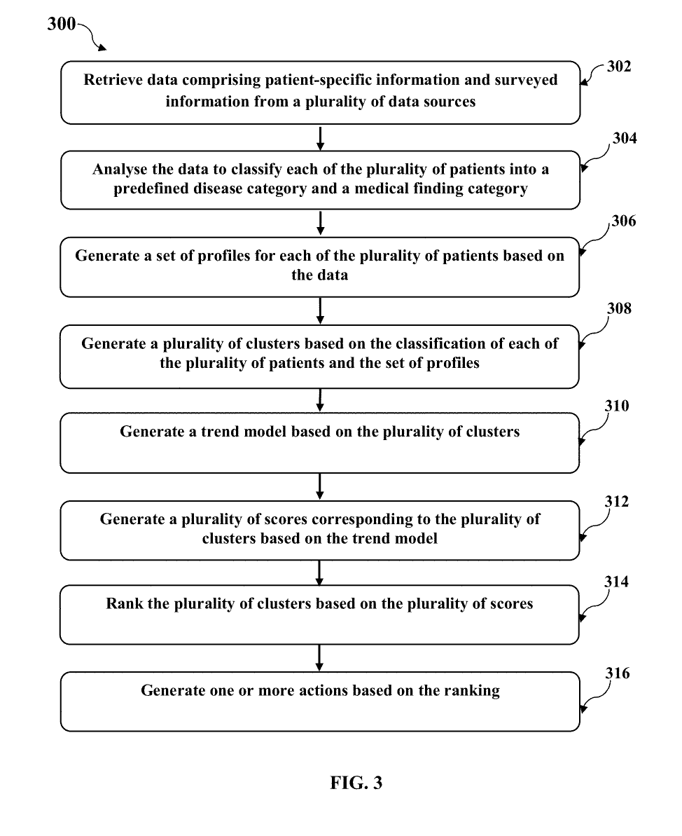

FIG. 3 shows a flowchart illustrating a method for generating one or more actions in a healthcare domain with some embodiments of the present disclosure.

As illustrated in FIG. 3, the method 300 comprises one or more blocks for generating one or more actions in the healthcare domain using a clinical action generating system 102. The method 300 may be described in the general context of computer executable instructions. Generally, computer executable instructions can include routines, programs, objects, components, data structures, procedures, modules, and functions, which perform particular functions or implement particular abstract data types.

The order in which the method 300 is described is not intended to be construed as a limitation, and any number of the described method blocks can be combined in any order to implement the method. Additionally, individual blocks may be deleted from the methods without departing from the scope of the subject matter described herein. Furthermore, the method can be implemented in any suitable hardware, software, firmware, or combination thereof.

At block 302, the clinical action generating system 102 retrieves data comprising patient-specific information and surveyed information from a plurality of data sources. Further, the data is associated with a plurality of patients.

At block 304, the clinical action generating system 102 analyzes the data to classify each of the plurality of patients into a predefined disease category and a medical finding category.

At block 306, the clinical action generating system 102 generates a set of profiles for each of the plurality of patients based on the data retrieved from the plurality of sources.

At block 308, the clinical action generating system 102 generates a plurality of clusters based on the classification of each of the plurality of patients and the set of profiles. Further, each cluster comprises two or more patients having similarity in at least one of the predefined disease category, the medical finding category, and the set of profiles.

At block 310, the clinical action generating system 102 generates a trend model based on the plurality of clusters. The trend model comprises a trend of a plurality of diseases and a rate of recovery of the plurality of diseases based on existing procedures and existing medications associated with each of the plurality of the patients.

At block 312, the clinical action generating system 102 generates a plurality of scores corresponding to the plurality of clusters based on the trend model.

At block 314, the clinical action generating system 102 ranks the plurality of clusters based on the plurality of scores.

At block 314, the clinical action generating system 102 generates one or more actions based on the ranking. Further, the one or more actions comprise a new procedure and a new medication.

### Computer System

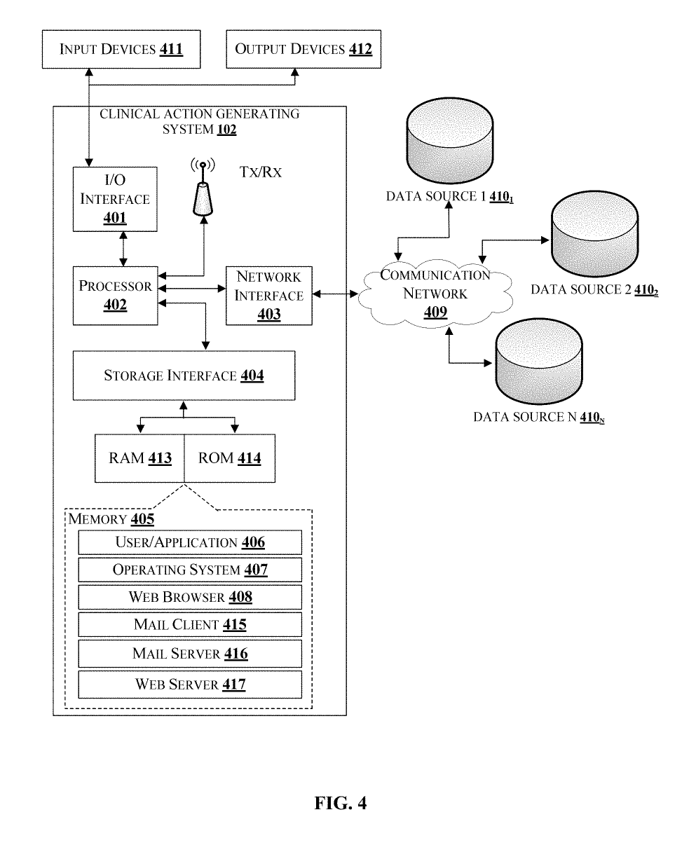

FIG. 4 illustrates a block diagram of an exemplary computer system 400 for implementing embodiments consistent with the present invention. In an embodiment, the computer system 400 can be the clinical action generating system 102 which is used for generating one or more actions in a healthcare domain. The computer system 400 may comprise a central processing unit (“CPU” or “processor”) 402. The processor 402 may comprise at least one data processor for executing program components for executing user- or system-generated business processes. The processor 402 may include specialized processing units such as integrated system (bus) controllers, memory management control units, floating point units, graphics processing units, digital signal processing units, etc.

The processor 402 may be disposed in communication with one or more input/output (I/O) devices (411 and 412) via I/O interface 401. The I/O interface 401 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc.

Using the I/O interface 401, the computer system 400 may communicate with one or more I/O devices (411 and 412).

In some embodiments, the processor 402 may be disposed in communication with a communication network 409 via a network interface 403. The network interface 403 may communicate with the communication network 409. The network interface 403 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. The communication network 409 can be implemented as one of the different types of networks, such as intranet or Local Area Network (LAN) and such within the organization. The communication network 409 may either be a dedicated network or a shared network, which represents an association of the different types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 409 may include a variety of network devices, including routers, bridges, servers, computing devices, storage devices, etc. Further, the one or more data sources 410₁ to 410n may include, but not limited to, clinical sources, hospice item set (HIS), legacy system, social media etc.

In some embodiments, the processor 402 may be disposed in communication with a memory 405 (e.g., RAM 413, ROM 414, etc. as shown in FIG. 4) via a storage interface 404. The storage interface 404 may connect to memory 405 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 405 may store a collection of program or database components, including, without limitation, user/application data 406, an operating system 407, web browser 408 etc. In some embodiments, computer system 400 may store user/application data 406, such as the data, variables, records, etc. as described in this invention. Such databases may be implemented as fault-tolerant, relational, scalable, secure databases such as Oracle or Sybase.

The operating system 407 may facilitate resource management and operation of the computer system 400. Examples of operating systems include, without limitation, Apple Macintosh OS X, UNIX, Unix-like system distributions (e.g., Berkeley Software Distribution (BSD), FreeBSD, Net BSD, Open BSD, etc.), Linux distributions (e.g., Red Hat, Ubuntu, K-Ubuntu, etc.), International Business Machines (IBM) OS/2, Microsoft Windows (XP, Vista/7/8, etc.), Apple iOS, Google Android, Blackberry Operating System (OS), or the like. I/O interface 401 may facilitate display, execution, interaction, manipulation, or operation of program components through textual or graphical facilities. For example, I/O interface may provide computer interaction interface elements on a display system operatively connected to the computer system 400, such as cursors, icons, check boxes, menus, windows, widgets, etc. Graphical User Interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

In some embodiments, the computer system 400 may implement a web browser 408 stored program component. The web browser may be a hypertext viewing application, such as Microsoft Internet Explorer, Google Chrome, Mozilla Firefox, Apple Safari, etc. Secure web browsing may be provided using Secure Hypertext Transport Protocol (HTTPS) secure sockets layer (SSL), Transport Layer Security (TLS), etc. Web browsers may utilize facilities such as AJAX, DHTML, Adobe Flash, JavaScript, Java, Application Programming Interfaces (APIs), etc. In some embodiments, the computer system 400 may implement a mail server stored program component. The mail server may be an Internet mail server such as Microsoft Exchange, or the like. The mail server may utilize facilities such as Active Server Pages (ASP), ActiveX, American National Standards Institute (ANSI) C++/C#, Microsoft .NET, CGI scripts, Java, JavaScript, PERL, PHP, Python, WebObjects, etc. The mail server may utilize communication protocols such as Internet Message Access Protocol (IMAP), Messaging Application Programming Interface (MAPI), Microsoft Exchange, Post Office Protocol (POP), Simple Mail Transfer Protocol (SMTP), or the like. In some embodiments, the computer system 400 may implement a mail client stored program component. The mail client may be a mail viewing application, such as Apple Mail, Microsoft Entourage, Microsoft Outlook, Mozilla Thunderbird, etc.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

### Advantages of the Embodiment of the Present Disclosure are Illustrated Herein

In an embodiment, the present disclosure provides a method for generating one or more actions (i.e., clinical actions) based on multiple dimensions and features.

In an embodiment, the method of present disclosure provides extraction of unseen/unknown categories or classes which are not even specified in medical ontology.

In an embodiment, the method of present disclosure provides better filtering technique while retrieving the data from the multiple data sources.

In an embodiment, the method of present disclosure enables the system to correlate results with different procedures and medication applied on a particular section of the patients. Thus, the system provides medical recommendations based on best possible success rate.

In an embodiment, the method of present disclosure provides customized treatment plan for an individual patient.

In an embodiment, the method of present disclosure also enables the medical practitioner to perform a search based on which the system generates the recommendations.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The enumerated listing of items does not imply that any or all of the items are mutually exclusive, unless expressly specified otherwise.

The terms “a”, “an” and “the” mean “one or more”, unless expressly specified otherwise.

A description of an embodiment with several components in communication with each other does not imply that all such components are required. On the contrary a variety of optional components are described to illustrate the wide variety of possible embodiments of the invention.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for generating one or more actions in a healthcare domain, the method comprising:
retrieving, by a clinical action generating system, data comprising patient-specific information and surveyed information from a plurality of data sources, wherein the data is associated with a plurality of patients;
analyzing, by the clinical action generating system, the data to classify each of the plurality of patients into a predefined disease category and a medical finding category;
generating, by the clinical action generating system:
a set of profiles for each of the plurality of patients based on the data,
a plurality of clusters based on the classification of each of the plurality of patients and the set of profiles, wherein each cluster comprises two or more patients having similarity in at least one of the predefined disease category, the medical finding category, or the set of profiles,
a trend model based on the plurality of clusters, wherein the trend model comprises a trend of a plurality of diseases and a rate of recovery of the plurality of diseases based on existing procedures and existing medications associated with each of the plurality of the patients, and
a plurality of scores corresponding to the plurality of clusters based on the trend model;
ranking, by the clinical action generating system, the plurality of clusters based on the plurality of scores; and
generating, by the clinical action generating system, one or more actions based on the ranking, wherein the one or more actions comprises a new procedure and a new medication.

2. The method as claimed in claim 1, wherein the plurality of data sources comprises at least one of clinical sources, hospice item set (HIS), legacy system, social media, blogs or journals.

3. The method as claimed in claim 1, wherein:
the patient-specific information comprises at least one of diagnosis summary, health parameters or results of a medical test conducted upon the patient, and
the surveyed information comprises at least one of sample size of a population, criticality of patients, post-medication observations or other findings by the research practitioners and doctors.

4. The method as claimed in claim 1 further comprising building, by the clinical action generating system, a learning model based on the patient-specific information and the surveyed information.

5. The method as claimed in claim 1 further comprising a data exchange policy for exchanging the patient-specific information between a plurality of devices associated with a plurality of entities, wherein the plurality of entities indicates healthcare institutions.

6. The method as claimed in claim 1 further comprising extracting, by the clinical action generating system, etiological features from the data, wherein the etiological features indicate one or more causes for the plurality of diseases present in the predefined disease category.

7. The method as claimed in claim 1, wherein the set of profiles comprises at least one of a persona, a family profile or a genetic profile.

8. The method as claimed in claim 1 further comprising creating, by the clinical action generating system, a knowledge database based on the data retrieved, the set of profiles, the plurality of clusters and the trend model.

9. The method as claimed in claim 1 further comprising:
receiving, by the clinical action generating system, a query from a user, wherein the query is formed using one or more Boolean operators;
executing, by the clinical action generating system, the query to retrieve two or more clusters from the plurality of clusters stored in the knowledge database, wherein the two or more clusters retrieved are related to each other; and
correlating, by the clinical action generating system, the two or more clusters to generate one or more correlated-clusters, wherein the one or more correlated-clusters is generated based on at least one of symptoms, observations, procedures, medication outcomes, rate of change of test parameters or health parameters.

10. A clinical action generating system for generating one or more actions in a healthcare domain, the system comprising:
a processor; and
a memory communicatively coupled to the processor, wherein the memory stores processor-executable instructions, which, on execution, causes the processor to:
retrieve data comprising patient-specific information and surveyed information from a plurality of data sources, wherein the data is associated with a plurality of patients;
analyze the data to classify each of the plurality of patients into a predefined disease category and a medical finding category;
generate:
a set of profiles for each of the plurality of patients based on the data,
a plurality of clusters based on the classification of each of the plurality of patients and the set of profiles, wherein each cluster comprises two or more patients having similarity in at least one of the predefined disease category, the medical finding category, or the set of profiles,
a trend model based on the plurality of clusters, wherein the trend model comprises a trend of a plurality of diseases and a rate of recovery of the plurality of diseases based on existing procedures and existing medications associated with each of the plurality of patients, and
a plurality of scores corresponding to the plurality of clusters based on the trend model;
rank the plurality of clusters based on the plurality of scores; and
generate one or more actions based on the ranking, wherein the one or more actions comprises a new procedure and a new medication.

11. The clinical action generating system as claimed in claim 10, wherein the plurality of data sources comprises at least one of clinical sources, hospice item set (HIS), legacy system, social media, blogs or journals.

12. The clinical action generating system as claimed in claim 10, wherein:
the patient-specific information further comprises at least one of diagnosis summary, health parameters or results of a medical test conducted upon a patient, and
the surveyed information comprises at least one of sample size of a population, criticality of patients, observations been made after certain medication or other findings by the research practitioners and doctors.

13. The clinical action generating system as claimed in claim 10, wherein the processor is further configured to build a learning model based on the patient-specific information and the surveyed information.

14. The clinical action generating system as claimed in claim 10, wherein the processor further facilitates a data exchange policy for exchanging the patient-specific information between a plurality of devices associated with a plurality of entities, wherein the plurality of entities indicates healthcare institutions.

15. The clinical action generating system as claimed in claim 10, wherein the processor is further configured to extract etiological features from the data, wherein the etiological features indicate one or more causes for the plurality of diseases present in the predefined disease category.

16. The clinical action generating system as claimed in claim 10, wherein the set of profiles comprises at least one of a persona, a family profile or a genetic profile.

17. The clinical action generating system as claimed in claim 10, wherein the processor is further configured to create a knowledge database based on the data retrieved, the set of profiles, the plurality of clusters and the trend model.

18. The clinical action generating system as claimed in claim 10, wherein the processor is further configured to:
receive a query from a user, wherein the query is formed using one or more Boolean operators;
execute the query to retrieve two or more clusters from the plurality of clusters stored in the knowledge database, wherein the two or more clusters retrieved are related to each other; and
correlate the two or more clusters to generate one or more correlated-clusters, wherein the one or more correlated-clusters is generated based on at least one of symptoms, observations, procedures, medication outcomes, rate of change of test parameters or health parameters.

19. A non-transitory computer-readable medium storing instructions for generating one or more actions in a healthcare domain wherein upon execution of the instructions by one or more processors, the processors perform operations comprising:
retrieving data comprising patient-specific information and surveyed information from a plurality of data sources, wherein the data is associated with a plurality of patients;
analyzing the data to classify each of the plurality of patients into a predefined disease category and a medical finding category;
generating:
a set of profiles for each of the plurality of patients based on the data,
a plurality of clusters based on the classification of each of the plurality of patients and the set of profiles, wherein each cluster comprises two or more patients having similarity in at least one of the predefined disease category, the medical finding category, or the set of profiles,
a trend model based on the plurality of clusters, wherein the trend model comprises a trend of a plurality of diseases and a rate of recovery of the plurality of diseases based on existing procedures and existing medications associated with each of the plurality of patients, and
a plurality of scores corresponding to the plurality of clusters based on the trend model;
ranking the plurality of clusters based on the plurality of scores; and
generating one or more actions based on the ranking, wherein the one or more actions comprises a new procedure and a new medication.

20. The medium as claimed in claim 19, the patient-specific information further comprises:
at least one of diagnosis summary, health parameters or results of a medical test conducted upon a patient, and
the surveyed information comprises at least one of sample size of a population, criticality of patients, observations been made after certain medication or other findings by the research practitioners and doctors.
